(19) United States
(12) Patent Application Publication
(10) Pub. No.: US 2008/0249398 A1
(43) Pub. Date: Oct. 9, 2008

(54) Hybrid Source Containing Multi-Radionuclides for Use in Radiation Therapy

(76) Inventors: George Fred HARDER, Lutz, FL (US); Max TAGHIZADEH, Palm Harbor, FL (US)

Correspondence Address: CARLTON FIELDS, P.A.;Attn: IP Dept., P.O. BOX 3239, TAMPA, FL, 33601-3239, US

(21) Appl. No.: 12/100,111

(22) Filed: Apr. 9, 2008

Related U.S. Application Data
(60) Provisional application No. 60/922,467, filed on Apr. 9, 2007.

(51) Int. Cl. A61M 36/12; A61B 6/12

(52) U.S. Cl. 600/424; 600/8

(57) ABSTRACT

A hybrid multi-radionuclide sealed source for use in brachytherapy comprising a plurality of radionuclides is disclosed. The differing decay rates of the radionuclides in the hybrid multi-radionuclide sealed source combine a large initial dose of radiation followed by an extended dose of radiation contained within the single source. The sealed source may comprise a seed, a flexible strand, a rigid strand, a wire, a coil or a catheter.

### CROSS REFERENCE TO RELATED APPLICATIONS

This application claims priority to U.S. Provisional Patent Appln. Ser. No. 60/922,467, filed Apr. 9, 2007 and titled “Hybrid Brachytherapy Sources,” the contents of which are herein incorporated by reference in their entirety.

### BACKGROUND OF THE INVENTION

Brachytherapy is a form of radiotherapy for patients suffering from a variety of conditions, such as prostate cancer, cervical cancer, endometrial cancer and cancers of the head and neck, as well as coronary artery disease. Additionally, brachytherapy may be used to provide a benefit to a patient such as serving as a marker or providing pain relief.

In brachytherapy, a sealed source (such as a seed, a flexible strand, a rigid strand, a wire, a coil or a catheter) containing radioactive isotopes (also referred to herein as radioisotopes or radionuclides) is placed inside or close to an area in or on a patient. Also referred to as endocurietherapy, five known types of brachytherapy include mold brachytherapy, strontium plaque therapy, interstitial brachytherapy, intracavitary brachytherapy and intravascular brachytherapy. Patients are typically human, but this approach can be used for any animal that suffers from these or similar conditions that are treatable using radiotherapy and brachytherapy in particular.

In mold brachytherapy, sealed sources containing radioactive seeds are placed close to the skin to treat superficial tumors.

Strontium plaque therapy is a surface application for very superficial lesions.

Interstitial radiation therapy is the direct implantation, either permanent or temporary, of radioactive substances into or near a tumor or other site to be treated.

In intracavitary brachytherapy, a source containing radionuclides is placed inside a naturally occurring body cavity. This type of treatment has been known to be used on such conditions as cancers of the cervix, uterus, esophageal and lung.

Intravascular brachytherapy uses radiation therapy to keep blocked heart arteries open by placing a wire (or seed train or ribbon) containing radionuclides inside a catheter placed inside an artery to deliver the radionuclides to the treatment site. Such treatment has been found to help keep blockages from forming in stents caused by in-stent restinosis.

Additionally, other forms of brachytherapy are under development today.

A variety of radionuclides have been used for brachytherapy, depending on the type of therapy involved. For example, strontium plaque therapy uses strontium-90. Other radionuclides used for different brachytherapy techniques include radium-226, gold-198, strontium-90, iodine-125, palladium-103, cesium-131, iridium-192, americium-241, yttrium-90, phosphorous-32, indium-114, indium-114m and more. Three radionuclides—iodine-125, palladium-103 and cesium-131—make up the majority of the current market of brachytherapy seeds.

Permanently implanted sealed sources, such as seeds, are typically constructed of tissue-inert material, such as titanium. Temporary treatments may be accomplished by delivering the radioactive sealed source, such as a seed, by way of injecting seeds with a tether to retrieve them after the dose has been delivered or by temporarily securing the seeds on the tumor or other site to be provided the benefit of radiation therapy.

Dose.

The dose, or amount of radioactive energy deposited into the tissue from the sealed source, is directly related to the radionuclide's half-life. The rate at which a radionuclide transmutes to another atom defines the half-life of the radionuclide. Shorter half-life radionuclides emit their energy and become inert faster than longer half-life radionuclides. For different radionuclides to deliver equivalent doses of radiation, a larger quantity of a short half-life radionuclide is needed as compared to a longer half-life radionuclide. Shorter half-life radionuclides, such as palladium-103, are believed to be more effective in treating aggressive, fast growing tumor cells where longer half-life radionuclides, such as iodine-125, are thought to be more effective on slower growing tumors.

Shorter half-life radionuclides require larger initial quantities of the radionuclide on the implant date in order to deliver a similar dose as longer half-life radionuclides. Larger quantities and smaller half-lives result in rapid and increased dose in surrounding tissue that can overwhelm cancerous tissue but can also overwhelm healthy tissue. The result can be discomfort to the patient from damage to periphery tissue that may require constructive therapy to repair.

Heterogeneity and Misdiagnosis

Cancer can be a heterogeneous combination of cells with different doubling times. The shorter the doubling time, the more aggressive the tumor. Misdiagnosis of the aggressiveness of the tumor can result in implantation of a less than effective single radionuclide. The hybrid multi-radionuclide sealed source compensates for misdiagnosis and heterogeneous tumors.

Tissue Edema.

Tissue edema is often observed after multiple needles are used to implant sealed sources, such as seeds. Tissue can swell 30% to 100% of the pre-implant volume. Reduction of the edema by a factor of one-half can take from 4 to 25 days. This swelling distances the cancerous tissue from the implanted sealed sources as well as realigns placement of the sealed source. Additionally, as the tissue or gland shrinks back to pre-operative size, implanted sealed sources can migrate from their original position or orientation causing further irregularity in the treatment plan. The effect is a reduction of the optimal treatment plan.

Using sources with longer half-lives ensures sufficient energy is available when tissue returns to normal.

### EXISTING STATE OF THE ART

U.S. Pat. No. 3,351,049, issued on Nov. 7, 1967 to Lawrence, the contents of which are herein incorporated by reference, discloses a container having a radioactive seed disposed therein in a manner such that the radioisotope cannot migrate through the encapsulating medium, where the seed comprises a therapeutic amount of a radioisotope that is distributed on a carrier body. The container is constructed of a low atomic numbered metal, such as stainless steel alloy or titanium, or in another embodiment, in an aluminum alloy tube sealed in an inert overcoating or container of plastic, ceramic or precious metal. The carrier body comprises a suitable material for collecting, concentrating and supporting the selected radioisotope and maintaining the radioisotope in a distributed form throughout the container. Lawrence teaches that the dose distribution in the tissue being treated is determined according to the location of the seeds, which allows for insufficiently treated areas to be treated by other means. The low atomic number materials can incorporate means to block x-rays to allow the positions of the seeds to be detected. Accordingly, one embodiment provides a small ball of a dense, high-atomic number material positioned midway in the seed. Another embodiment provides a wire of a high atomic number material located centrally in the axis of symmetry of the container. Placement of the high atomic number material within the container is important to avoid attenuation of the radiation. Placement is also important to permit the radiation to emit from the seed without encountering the high atomic number material as well as to permit a uniform radiation pattern to emit from the seed.

U.S. Pat. No. 4,323,055, issued on Apr. 6, 1982 to Kubiatowicz, the contents of which are herein incorporated by reference, discloses a radioactive iodine seed, where the container comprises a carrier body along which the radioactive iodine is distributed, wherein the carrier body is detectable by x-ray. Kubiatowicz further teaches that the invention permits both the location and the orientation of the seed in the tissue to be determined by x-ray.

U.S. Pat. No. 4,510,924, issued on Apr. 16, 1985 to Gray, the contents of which are herein incorporated by reference, discloses the employment of the radioisotope americium-241 in brachytherapy devices.

U.S. Pat. No. 4,702,228, issued on Oct. 27, 1987 to Russell, Jr. et. al., the contents of which are herein incorporated by reference, discloses a method for creating seeds containing palladium-102 which are then exposed to high electron flux to transmute a fraction of the palladium-102 to palladium-103.

U.S. Pat. No. 4,891,165, issued on Jan. 2, 1990 to Suthanthiran, the contents of which are herein incorporated by reference, discloses a capsule formed from two interfitting sleeves for retention of radioactive material for use in medical treatments.

U.S. Pat. No. 6,503,186, issued on Jan. 7, 2003 to Cutrer, the contents of which are herein incorporated by reference, discloses a radioactive seed comprising a plurality of x-ray detectable markers that disclose the orientation of the seed when exposed to x-ray. The seed comprises at least one carrier body, which in turn comprises a plurality of carrier units each impregnated with the radioisotope and distributed uniformly in the seed. The x-ray detectable markers are distributed among the carrier units so that the markers disclose the orientation of the seed when exposed to x-ray. The x-ray detectable markers may have different configurations to identify the particular type and dosage level of the radioactive source in the seed.

U.S. Pat. No. 6,712,752, issued on Mar. 30, 2004, and U.S. Pat. No. 7,172,549, issued on Feb. 6, 2007, both to Slater et. al., the contents of which are herein incorporated by reference, discloses the use of a plurality of radioactive therapeutic seeds wherein different seeds are provided with markers of different configurations to indicate their respective levels of radioactivity or half-life.

U.S. Pat. No. 6,793,798, issued on Sep. 21, 2004 to Chan et. al., the contents of which are herein incorporated by reference, discloses radioactively coated medical devices for use as stents, catheters, seeds, prostheses, valves, staples and other wound closure devices.

U.S. Pat. No. 6,986,880, issued on Jan. 17, 2006 to Coniglione et. al., the contents of which are herein incorporated by reference, discloses a radioactive composite as a therapeutic source for brachytherapy that comprises a polymeric matrix and a radioactive powder.

U.S. Pat. No. 7,244,226, issued on Jul. 17, 2007 to Terwilliger et. al., the contents of which are herein incorporated by reference, discloses a bio-absorbable delivery system for interstitial radioactive seeds. The radioactive seeds are placed into a fixture that spaces the seeds according to a predetermined plan, which is then encapsulated into a bio-absorbable polymer to form a flexible elongated member for insertion into tumors. The elongated member may be made echogenic by suspending minute air bubbles in the polymer.

U.S. Pat. No. 7,311,655, issued on Dec. 25, 2007 to Schaart, the contents of which are herein incorporated by reference, discloses a brachytherapy source material comprising indium-114m in radioactive equilibrium with its decay product, indium-114. Indium-114m is produced by irradiating In-113 with neutrons.

U.S. Pat. No. 7,316,644, issued on Jan. 8, 2008 to Bray, the contents of which are herein incorporated by reference, discloses a method of preparing Cesium-131 as a dispersed isotope which can be used in brachytherapy.

U.S. Pat. No. 7,322,928, issued on Jan. 29, 2008 to Reed et. al., the contents of which are herein incorporated by reference, discloses a radioactive member for use in brachytherapy comprising a hollow elongate bioabsorbable suture with radioactive seeds. The coloration allows easy identification of components inside the suture member.

### SUMMARY OF THE INVENTION

The invention relates to sealed sources for use in brachytherapy and other types of radiation therapy comprising multiple radionuclides in a single sealed source. Such sealed sources are useful in, but not limited to, permanent or temporary implantation into or close to a tumor site or other site in order to provide a benefit to a patient. The patient may be a human or may be any other animal having a condition that can be provided a benefit using radiotherapy, and in particular brachytherapy.

By combining multiple radionuclides into a single source, it is believed that hybrid multiple radionuclide sealed sources of the invention will compensate for misdiagnosis and heterogeneous tumors. Cancer can be a heterogeneous combination of cells with different doubling times. The shorter the doubling time, the more aggressive the tumor. Misdiagnosis of the aggressiveness of the tumor can result in implantation of a less than effective radionuclide.

By combining multiple radionuclides into a single source, it is believed that hybrid multiple radionuclide sealed sources may be manufactured which comprise reduced amounts of shorter half-life radionuclides but provide equivalent doses of radiation therapy, as compared to present technology sealed sources. The reduced amounts of shorter half-life radionuclides are believed to lessen or prevent overwhelming healthy tissue that typically results in periphery tissue damage while still combating aggressive cancerous tissues.

Hybrid multi-radionuclide sealed sources of the invention can be manufactured according to presently known or later developed techniques.

The invention also relates to an innovative technique for treating tumors or other conditions in patients treatable by, or otherwise provide a benefit to a patient through, radiation therapy and in particular brachytherapy, comprising the use of one or more hybrid multi-radionuclide sealed sources. It is believed that the greater initial radioactive strength of one or more short half-life radionuclides in combination with the sustained radioactive strength of one or more longer half-life radionuclides in a single sealed source synergistically combines the benefits of each radionuclide while reducing the detriments of each radionuclide. Further, the multi-radionuclide sealed sources of the invention permit the placement of such sealed sources comprising both short half-life radionuclides and longer half-life radionuclides at the same distance from the area to be treated by or to receive the benefit from radiation therapy. Multiple hybrid multi-radionuclide sealed sources may comprise an assembly of similar or dissimilar hybrid multi-radionuclide sealed sources of the invention.

The combination of short half-life radionuclides and long half-life radionuclides in a single sealed source is believed to improve the efficacy of brachytherapy by providing an initial higher dose to more aggressive tissues but also to sustain a lower but consistent dose over a longer period of time for tissue more distant from the sealed source. The invention permits the short half-life radionuclides and long half-life radionuclides to remain in a constant relationship with respect to each other to improve the efficacy of such treatment, as compared to present treatment with separate sealed sources of short half-life radionuclides and long half-life radionuclides.

Additionally, since tissue initially swells after implantation of seeds, a viable dose from the hybrid multi-radionuclide sealed source, after delivering its initial dose from the short half-life radionuclide, still has a radiation dose available for the treatment of tissue as that tissue returns to normal size within a few weeks.

Other features and advantages of the invention will become apparent from the description of the preferred embodiments in conjunction with the following figures.

### BRIEF DESCRIPTION OF THE DRAWINGS

The invention will be described with reference to the accompanying drawings, in which like elements are referenced with like numerals.

FIG. 1 depicts a first half-life for a hybrid multi-radionuclide sealed source whose total activity is comprised of 30% Pd-103 and 70% I-125.

FIG. 2 depicts a first half-life for a hybrid multi-radionuclide sealed source whose total activity is comprised of 50% Pd-103 and 50% I-125.

FIG. 3 depicts a first half-life for a hybrid multi-radionuclide sealed source whose total activity is comprised of 70% Pd-103 and 30% I-125.

FIG. 4 depicts one embodiment of a hybrid multi-radionuclide sealed source according to the invention.

FIG. 5 depicts another embodiment of a hybrid multi-radionuclide sealed source according to the invention.

FIG. 6 depicts another embodiment of a hybrid multi-radionuclide sealed source according to the invention.

### DETAILED DESCRIPTION OF THE INVENTION

Interstitial radiation brachytherapy places radioactive emissions in very close or direct contact with a tumor or other area to be treated, or in a patient to provide a benefit through radiation therapy, either permanently or temporarily. The permanent or temporary implantation of small, radioactive sealed sources into or near a tumor site or other area to be treated with, or to be provided a benefit through, radiation has been an effective method of therapy for decades. Brachytherapy is an effective alternative to surgical removal of cancerous tissue. The methods of implantation of radioactive sealed sources have improved over time, reducing the discomfort to the patient while increasing the effectiveness of treatment or other benefit.

A variety of radionuclides have been used for interstitial radiation therapy, including radium-226, gold-198, strontium-90, iodine-125, palladium-103, cesium-131, iridium-192, americium-241, yttrium-90, phosphorous-32, indium-114, indium-114m and more.

The current state of the art typically concentrates on three radionuclides—iodine-125, palladium-103 and cesium-131—which make up the majority of the current market of brachytherapy seeds.

Treatment.

Once a treatment plan of a tumor or provision of a benefit through radiation therapy using radioactive sealed sources has been determined for a patient, radioactive sealed sources are implanted in or provided on or in close proximity to the patient in accordance with the treatment plan specifically tailored to that patient and the patient's condition to be treated or benefit to be provided. For example, in the treatment of cancers, tumor cells are insulted by the photons created by the radioactive decay of the material within the seed. A rapid succession of photons causes minute damage to cells that eventually overwhelms the cell's ability to repair itself.

Two mechanisms cause cell death. The first mechanism is the overwhelming energy deposited into the cell by radioactive decay. The entire cell structure becomes damaged beyond its ability to repair. The second mechanism is through damage of the cell's DNA. Once damaged, the cell has four options: repair and remain viable, repair but not duplicate, mutate or immediate death. Repeated insults to the DNA will eventually produce cell death.

Other benefits through radiation therapy can also be provided through such radioactive sealed sources. For example, radioactive sealed sources can act as markers detectable by x-ray, ultrasound, MRI or other imaging techniques, or can provide pain relief.

Dose.

As a radionuclide releases energy, this energy is deposited into the local tissue. The amount of deposited energy is known as the dose. The treatment plan will stipulate a predetermined dose of radiation to be provided using the implanted sealed source. The dose is similar for each radionuclide, but the rate at which the dose is applied is directly related to the half-life of the specific radioactive material contained in the sealed source.

The rate at which a radionuclide transmutes from one isotope to another determines the half-life of the radionuclide. Stated differently, the half-life is that time when one-half of the original isotope decays away. Shorter half-life radionuclides emit their energy and transmute to another isotope at a faster rate. As the energy is released at this faster rate, the dose to the tissue also occurs at a faster rate. To impart the same dose to tissue, larger amounts of a shorter half-life radionuclide are needed as compared to the amounts of a longer half-life radionuclide.

By depositing a dose faster, shorter half-life radionuclides are believed to be more effective on aggressive, fast growing tumor cells by overwhelming and destroying cells. Longer half-life radionuclides release their dose and affect tumor cells over an extended period of time. This improves the ability to affect the DNA and cause cell invalidation or death. However, the longer half-life radionuclides may not have the initial dose impact to affect aggressive tumor cells and some cells may escape treatment. For a heterogeneous tumor with both aggressive and non-aggressive cells or for a misdiagnosed treatment, a single radionuclide seed may be inappropriate.

The hybrid multi-radionuclide sealed source of the invention combines the benefits of multiple types of radionuclides to treat both aggressive and non-aggressive tumors, as well as to provide other benefits through radiation therapy. Such radioactive sealed sources may include, according to the invention, high activity, short half-life radionuclides which can treat fast growing cells in combination with the sustained dose from a long half-life radionuclide which can treat less aggressive or more distant tumor cells.

Half-Life.

The half-life for each radionuclide is a known, constant value. At each half-life interval, one-half of the remaining radionuclide has decayed, meaning that half the remaining dose has been delivered. Therefore, after one half-life, 50% of the radionuclide remains. After a second half-life 25% of the initial radionuclide remains. After a third half-life 12.5% of the initial radionuclide remains. For any single radionuclide, this pattern is constant and predictable.

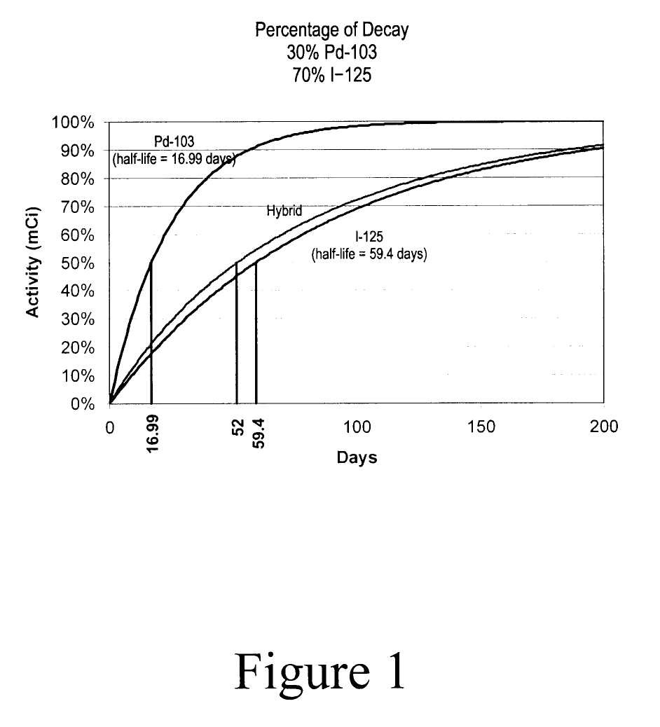

The combination of two or more radionuclides alters the concept of half-life. FIGS. 1-3 demonstrate how differing combinations of iodine-125 and palladium-103 in a hybrid multi-radionuclide sealed source can result in different half-lives for a sealed source containing those two radionuclides. These examples are intended merely as illustrations of the invention and not as a limitation on the invention.

The half-life for any combination of two or more radionuclides will always fall between the longest and shortest half-lives of any combination of radionuclides. Each radionuclide contributes a portion of the overall dose in relation to the percentage of that radionuclide remaining. The shorter half-life radionuclide initially provides the larger portion of the radiation dose provided by the combined radionuclides. Over time, the shorter half-life radionuclide decays away and the longer half-life radionuclides become the predominant contributors to the radiation dose of the combined radionuclides. The half-life of the hybrid sealed source containing the combined radionuclides thus will be greater than the shortest half-life and then slowly tend toward the half-life of the longest half-life radionuclide over time.

To determine the first half-life of such a multi-radionuclide sealed source, the activity of each individual radionuclide must be derived. Two methods for determining the initial half-life of a multi-radionuclide sealed source are provided below. Other methods, either presently known or later developed, may be used to make this determination.

Method 1 for Calculating Radionuclide Activity of a Hybrid Multi-Radionuclide Sealed Source Using Differences in Half-Life.

For two radionuclides with different half-lives, two successive readings of the total activity can be used to calculate the initial activity of each radionuclide, using the following equations:

$${A}_{0} = {A}_{L} + {A}_{S}$$

$${A}_{1} = {A}_{L} {e}^{- {λ}_{L} t} + {A}_{S} {e}^{- {λ}_{S} t}$$

$${A}_{1} = ( {A}_{0} - {A}_{S} ) {e}^{- {λ}_{L} t} + {A}_{S} {e}^{- {λ}_{S} t}$$

$${A}_{1} = {A}_{0} {e}^{- {λ}_{L} t} + {A}_{S} ( {e}^{- {λ}_{S} t} - {e}^{- {λ}_{L} t} )$$

$${A}_{S} = \frac{{A}_{1} - {A}_{0} {e}^{- {λ}_{L} t}}{{e}^{- {λ}_{S} t} - {e}^{- {λ}_{L} t}}$$

### Where:

A₀ is the initial measured activity of the hybrid sealed source;
A₁ is the measured activity of the hybrid sealed source at some future time;
A_{L} is the unknown activity of the long half-life radionuclide;
A_{S} is the unknown activity of the short half-life radionuclide;
λ_{L} is the long half-life radionuclide's decay constant; and
λ_{S} is the short half-life radionuclide's decay constant.

Method 2 for Calculating Radionuclide Activity of a Hybrid Multi-Radionuclide Sealed Source Using Decay Energies.

To illustrate this method, a hybrid multi-radionuclide sealed source comprising palladium-103 and iodine-125 will be used. This is not intended in any manner to limit the invention and is used for illustration purposes only. Persons of ordinary skill in the art can adapt this procedure to any combination of radioactive sources without undue experimentation.

Palladium-103 and Iodine-125 have predominant gamma energies below 30 keV when the radionuclide decays. Palladium-103, however, has one gamma energy at 357 keV with an abundance of 0.0221 percent. Abundance is that percent of emissions from the decay of the radionuclide that occur at the specified energy. A calibrated palladium-103 seed is placed in a gamma spectroscopy system. After a sufficient time to ensure good resolution of the 357 keV gamma energy, the specific activity is used to calibrate the gamma spectroscopy system. This can be done in one of two ways. The system software can be adjusted to read the correct activity or the displayed activity can be used in conjunction with the calibrated activity to create a correction factor by dividing the measured counts per second by the seed's actual activity.

Using a gross gamma well chamber, a calibrated iodine-125 sealed source is placed inside using the palladium-103 calibration setting. A correction factor for iodine-125 is calculated using the sealed source's activity divided by the displayed activity.

A sealed source, containing both palladium-103 and iodine-125, is placed in the gamma spectroscopy system and the activity of the palladium-103, using the 357 keV energy peak, is measured. The sealed source is then placed in the gross gamma well chamber using the palladium-103 calibration setting. The activity determined using the gamma spectroscopy system is subtracted from the displayed activity. The remaining activity is the contribution by the iodine-125 and is multiplied by the correction factor derived earlier using the calibrated iodine-125 sealed source. The result is the activity of the iodine-125 in the hybrid sealed source.

The effective half-life of a sealed source containing two or more radionuclides is thus a function of the percentage of the initial activity of each radionuclide within the sealed source. For example, if palladium-103 and iodine-125 are combined with fifty percent of the initial total activity of the combined radionuclides coming from each radionuclide, the effective half-life will be approximately 44 days, as seen below in Table 1. At 44 days, 83 percent of the palladium-103 (half-life of 16.99 days) and 40 percent of the iodine-125 (half-life of 59.4 days) have decayed.

TABLE 1
Half-Life Calculations

| | |
|---|---|
| Percent Initial Activity | |
| From Each Radionuclide | Days to Decay to 50% |

| Pd-103 | I-125 | (1^{st} Half-Life) |
|---|---|---|
| 100 | 0 | 16.99 |
| 70 | 30 | 34 |
| 50 | 50 | 44 |
| 30 | 70 | 52 |
| 0 | 100 | 59.4 |

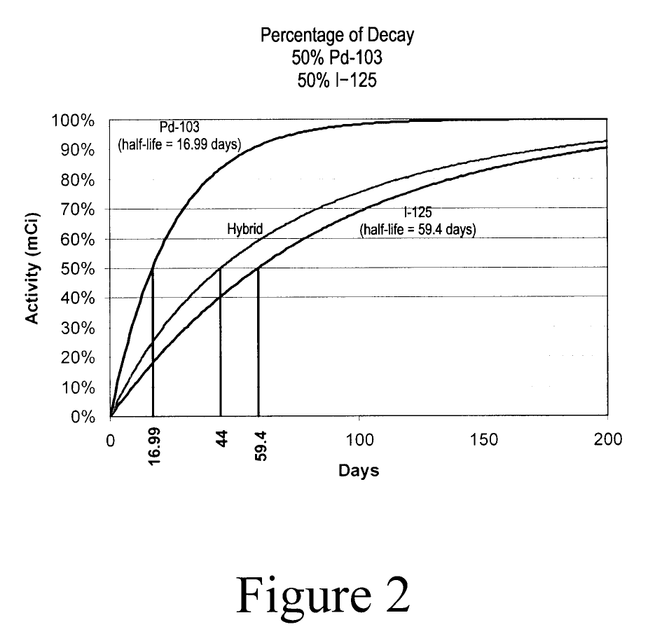

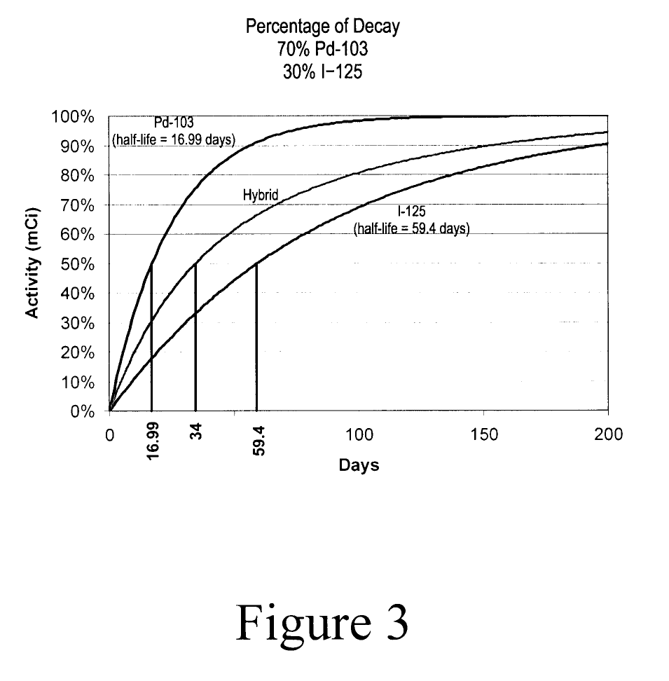

FIG. 1 depicts the decay pattern of pure palladium-103, pure iodine-125 and a combination of 30% initial activity from palladium-103 and 70% initial activity from iodine-125. FIG. 2 depicts the decay pattern of pure palladium-103, pure iodine-125 and a combination of 50% initial activity from palladium-103 and 50% initial activity from iodine-125. FIG. 3 depicts the decay pattern of pure palladium-103, pure iodine-125 and a combination of 70% initial activity from palladium-103 and 30% initial activity from iodine-125. As can be seen, as the palladium-103 provides increased amounts of initial activity to the combined radionuclides, the half-life of the combination decreases from 52 days for 30% palladium-103/70% iodine-125 to 34 days for 70% palladium-103/30% iodine-125.

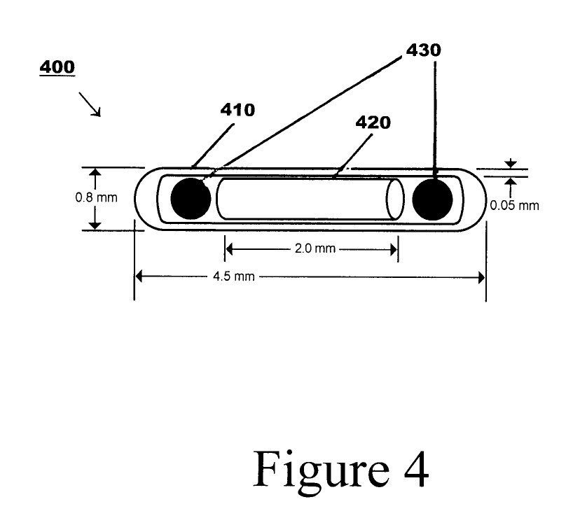

Turning now to construction of the sealed source, FIG. 4 depicts one embodiment of a hybrid multi-radionuclide sealed source 400 of the invention. In this embodiment, the hybrid multi-radionuclide sealed source 400 comprises a seed. The outer casing 410 of the seed 400 may comprise a titanium shell as currently known to be used for seeds. Other casings now known or later developed may be used as well, and the type of casing in no way limits the invention. Radioactive material is placed inside the casing 410 and sealed. Each seed 400 is verified to ensure the radioactive material is sealed within the casing. A typical casing 410 may have a wall thickness of about 0.05 mm where the casing 410 comprises titanium. A seed 400 constructed of titanium, as shown in FIG. 4, may typically be 4.5 mm long and approximately 0.8 mm in diameter. These dimensions may vary as known to those of ordinary skill in the art.

A silver rod 420 can be placed in the center of the seed 400 to serve the dual purpose of an x-ray marker and also a substrate for the application of a radionuclide, for example iodine-125. For a seed 400 constructed of a titanium casing 410 as shown in FIG. 4, the silver rod 420 may be approximately 2.0 mm in length and be of an overall dimension to fit within the casing 410.

As seen in Table 1, iodine-125 has a half-life of 59.4 days. In the following example, iodine-125 will be considered the radionuclide of the hybrid multi-radionuclide seed having the “longer” half-life. Other radionuclides could also be deposited on, absorbed in, adhered to or adsorbed on the silver rod 420 and serve as the longer half-life radionuclide. Other structures, such as spheres or other geometric shaped substrates or any other x-ray marker and substrate for a radionuclide now known or later developed, may be used in place of silver rod 420. Similarly, the mounting surface or absorbing medium for the radionuclide may be separate from the x-ray marker. Further, the seed 400 may not include a marker at all, or the marker may be one that is detectable by ultrasound, MRI or other imaging techniques.

On either end of the silver rod 420 of the embodiment of the invention depicted in FIG. 4 are one or more polystyrene spheres 430 which contain a second radionuclide, such as, in this example, palladium-103. As seen in Table 1, palladium-103 has a half-life of 16.99 days. In the following example, palladium-103 can be considered the radionuclide of the hybrid multi-radionuclide seed 400 having the “shorter” half-life. Other radionuclides, such as cesium-131, could also be absorbed in or adsorbed on the polystyrene spheres 430 to serve as the short half-life radionuclide. Any material now known or later developed can also be used in the hybrid multi-radionuclide seed of the invention to support the second radionuclide. The dimension of the polystyrene spheres 430 should be of a dimension to fit within the casing 410. Alternatively, polystyrene spheres 430 could contain different radionuclides, for example a second and a third radionuclide, each different from each other and also different from the first radionuclide. The polystyrene may also be of any shape with the only requirement of placement in the titanium casing 410.

Techniques for preparing such a seed 400 are known and can be prepared without undue experimentation by those of ordinary skill in the art. Further, the hybrid multi-radionuclide sealed source may comprise a flexible strand, a rigid strand, a coil, a catheter or any other sealed source now known or later developed.

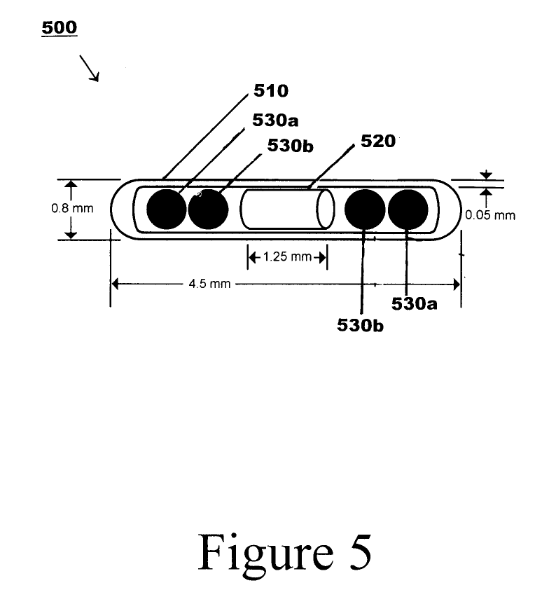

FIG. 5 depicts another embodiment of a hybrid multi-radionuclide sealed source 500 of the invention. As previously discussed with respect to FIG. 4, the hybrid multi-radionuclide sealed source 500 may comprise a seed constructed of a titanium casing 510 having similar dimensions as current single source seeds. The embodiment of FIG. 5 also comprises a silver rod 520 to support a radionuclide and to act as an x-ray marker. Again, other structures, such as spheres or other geometric shaped substrates or any other x-ray marker and substrate for a radionuclide now known or later developed, may be used in place of silver rod 520. Similarly, the mounting surface for the radionuclide may be separate from the x-ray marker. Further, the seed 500 may not include a marker at all, or the marker may be one that is detectable by ultrasound, MRI or other imaging techniques.

In the embodiment of FIG. 5, multiple polystyrene spheres 530ₐ and 530_{b} are contained in the seed 500, where polystyrene spheres 530ₐ may contain a different radionuclide from polystyrene spheres 530_{b}. The embodiment shown in FIG. 5 could provide a hybrid multi-radionuclide seed containing three different radionuclides, for example, iodine-125, palladium-103 and cesium-131.

Techniques for preparing such a seed 500 are known and can be prepared without undue experimentation by those of ordinary skill in the art. Further, the hybrid multi-radionuclide sealed source may comprise a flexible strand, a rigid strand, a coil, a catheter or any other sealed source now known or later developed.

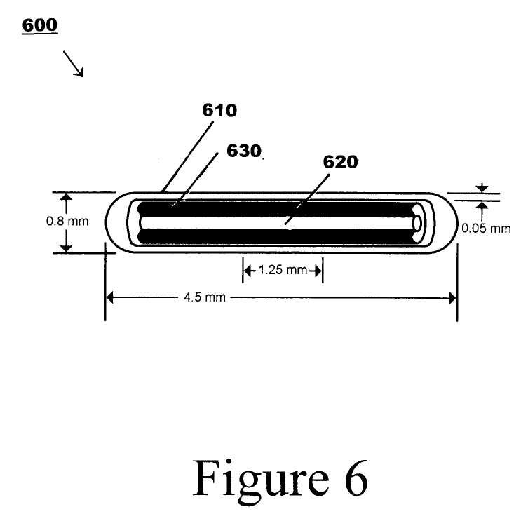

FIG. 6 depicts another embodiment of a hybrid multi-radionuclide sealed source 600 of the invention. As discussed with respect to FIG. 4, the hybrid multi-radionuclide sealed source 600 may comprise a seed that is constructed of a titanium casing 610 having similar dimensions as existing single source seeds. In this embodiment, the silver rod 620 has been lengthened and its diameter reduced compared to silver rods 420 and 520. Iodine-125 could be adhered on this elongated marker 620. Again, other structures, such as spheres or other geometric shaped substrates or any other x-ray marker and substrate for a radionuclide now known or later developed, may be used in place of silver rod 620. Similarly, the mounting surface for the radionuclide may be separate from the x-ray marker. Further, the seed 600 may not include a marker at all, or the marker may be one that is detectable by ultrasound, MRI or other imaging techniques.

A polystyrene or ceramic composite 630 containing one or more radionuclides could be deposited around this silver rod 620.

Techniques for preparing such a seed 600 are known and can be prepared without undue experimentation by those of ordinary skill in the art. Further, the hybrid multi-radionuclide sealed source may comprise a flexible strand, a rigid strand, a coil, a catheter or any other sealed source now known or later developed.

Although the examples recite the use of a hybrid multi-radionuclide seed as is commonly used in interstitial therapy, other methods of brachytherapy and radiotherapy could similarly use the hybrid multi-radionuclide sealed source. Other variations of the invention include radioactive sealed sources comprising flexible strands, rigid strands, polymeric casings, wires, coils or catheters. Further, inert materials may be placed in the sealed source and activated to a radioactive state after the source is sealed to provide the radionuclides.

The foregoing embodiments have been presented for the purpose of illustration and description only and are not to be construed as limiting the scope of the invention in any way. The scope of the invention is to be determined from the claims appended hereto.

What is claimed is:

1. A hybrid multi-radionuclide sealed source comprising:
a single sealed source comprising a plurality of radionuclides suitable for use in radiation therapy, each radionuclide having a different decay property from each other radionuclide in the single sealed source.

2. The hybrid multi-radionuclide sealed source of claim 1, further comprising a first radionuclide and a second radionuclide.

3. The hybrid multi-radionuclide sealed source of claim 2, wherein the hybrid multi-radionuclide sealed source is implanted into a patient to provide treatment for any combination of prostate cancer, cervical cancer, breast cancer, ophthalmic cancer, endometrial cancer, cancers of the head, cancers of the neck and coronary artery disease.

4. The hybrid multi-radionuclide sealed source of claim 3, further comprising a sealed source suitable for temporary placement of the multi-radionuclide in, on or in close proximity to the patient.

5. The hybrid multi-radionuclide sealed source of claim 3, further comprising a sealed source suitable for permanent placement of the multi-radionuclide seed in, on or in close proximity to the patient.

6. The hybrid multi-radionuclide sealed source of claim 2, wherein any of the first radionuclide and the second radionuclide is activated to its radioactive state following placement in the sealed source.

7. The hybrid multi-radionuclide sealed source of claim 2, wherein the first and second radionuclides are disposed in the sealed source by deposition, absorption, adsorption, adherence or any combination thereof.

8. The hybrid multi-radionuclide sealed source of claim 2, wherein the first radionuclide comprises one of radium-226, gold-198, strontium-90, iodine-125, palladium-103, cesium-131, iridium-192, americium-241, yttrium-90, phosphorous-32 or indium-114m.

9. The hybrid multi-radionuclide sealed source of claim 8, wherein the second radionuclide comprises one of radium-226, gold-198, strontium-90, iodine-125, palladium-103, cesium-131, iridium-192, americium-241, yttrium-90, phosphorous-32 or indium-114m.

10. The hybrid multi-radionuclide sealed source of claim 1, wherein the plurality of radionuclides comprises any combination of radium-226, gold-198, strontium-90, iodine-125, palladium-103, cesium-131, iridium-192, americium-241, yttrium-90, phosphorous-32 or indium-114m.

11. The hybrid multi-radionuclide sealed source of claim 10, wherein the hybrid multi-radionuclide sealed source is implanted into a patient to provide treatment for any combination of prostate cancer, cervical cancer, breast cancer, ophthalmic cancer, endometrial cancer, cancers of the head, cancers of the neck and coronary artery disease.

12. The hybrid multi-radionuclide sealed source of claim 11, further comprising a sealed source suitable for temporary placement of the multi-radionuclide in, on or in close proximity to the patient.

13. The hybrid multi-radionuclide sealed source of claim 11, further comprising a sealed source suitable for permanent placement of the multi-radionuclide seed in, on or in close proximity to the patient.

14. The hybrid multi-radionuclide sealed source of claim 10, comprising at least three radionuclides.

15. The hybrid multi-radionuclide sealed source of claim 14, wherein the hybrid multi-radionuclide sealed source is implanted into a patient to provide treatment for any combination of prostate cancer, cervical cancer, breast cancer, ophthalmic cancer, endometrial cancer, cancers of the head, cancers of the neck and coronary artery disease.

16. The hybrid multi-radionuclide sealed source of claim 15, further comprising a sealed source suitable for temporary placement of the multi-radionuclide in, on or in close proximity to the patient.

17. The hybrid multi-radionuclide sealed source of claim 15, further comprising a sealed source suitable for permanent placement of the multi-radionuclide seed in, on or in close proximity to the patient.

18. The hybrid multi-radionuclide sealed source of claim 1, further comprising a material adhered to the outer surface of the hybrid multi-radionuclide sealed source.

19. The hybrid multi-radionuclide sealed source of claim 1, wherein the sealed source comprises a seed, a container, a flexible strand, a rigid strand, a wire, a coil or a catheter.

20. The hybrid multi-radionuclide sealed source of claim 1, further comprising a marker detectable to determine at least one of the location and orientation of a radionuclide in the sealed source.

21. The hybrid multi-radionuclide sealed source of claim 20, wherein the marker is detectable by x-ray, ultrasound, MRI or other imaging techniques.

22. The hybrid multi-radionuclide sealed source of claim 21, further comprising a marker detectable to determine at least one of the location and orientation of each radionuclide in the sealed source.

23. An assembly comprising a plurality of hybrid multi-radionuclide sealed sources as claimed in claim 1.

24. The assembly of claim 23, wherein the plurality of radionuclides in at least one of the hybrid multi-radionuclide sealed sources differ from the plurality of radionuclides in at least one other of the hybrid multi-radionuclide sealed sources in the assembly.

25. A method of preparing a radiotherapy treatment plan including one or more hybrid multi-radionuclide sealed sources to provide a benefit to a patient through radiation therapy, comprising:
determining a radiotherapy treatment plan comprising a radiation dose designed to provide a benefit to a patient through radiation therapy;
determining a combination of one or more radioactive sources to provide the radiation dose wherein at least one of the radioactive sources comprises a hybrid multi-radionuclide seed as claimed in claim 1; and
preparing one or more hybrid multi-radionuclide sealed sources in accordance with the radiotherapy treatment plan.

26. The method of claim 25, wherein the benefit comprises medical therapy for any combination of prostate cancer, cervical cancer, endometrial cancer, breast cancer, ophthalmic cancer, cancers of the head, cancers of the neck, coronary artery disease, pain management and as a marker.

27. The method of claim 26, wherein the patient comprises a human.

28. A method of providing a medical benefit to a patient through radiation therapy, comprising placing one or more hybrid multi-radionuclide sealed sources as claimed in claim 1 near or in an area of a patient to provide a medical benefit.

29. The method of claim 28, wherein at least one hybrid multi-radionuclide sealed source is placed temporarily in, on or in close proximity to the patient.

30. The method of claim 28, wherein at least one hybrid multi-radionuclide sealed source is placed permanently in, on or in close proximity to the patient.
